(19) United States
(12) Patent Application Publication
(10) Pub. No.: US 2021/0346135 A1
(43) Pub. Date: Nov. 11, 2021

(54) Dental Mouthpieces

(71) Applicants: Amy Kerr, Hot Springs, SC (US); Adam Ontiveros, Orcutt, CA (US); Brian Halleck, Santa Barbara, CA (US); Jacob Swartz, Santa Barbara, CA (US); James Hirsch, Santa Barbara, CA (US); Shaun Valentine, Fort Thomas, KY (US); Yashdeep Bhatti, Santa Barbara, CA (US)

(72) Inventors: Amy Kerr, Hot Springs, SC (US); Adam Ontiveros, Orcutt, CA (US); Brian Halleck, Santa Barbara, CA (US); Jacob Swartz, Santa Barbara, CA (US); James Hirsch, Santa Barbara, CA (US); Shaun Valentine, Fort Thomas, KY (US); Yashdeep Bhatti, Santa Barbara, CA (US)

(21) Appl. No.: 17/383,258

(22) Filed: Jul. 22, 2021

Related U.S. Application Data
(63) Continuation of application No. 17/234,484, filed on Apr. 19, 2021.
(60) Provisional application No. 63/125,182, filed on Dec. 14, 2020.
(60) Provisional application No. 63/011,843, filed on Apr. 17, 2020.

(51) Int. Cl. A61C 17/10; A61C 17/06

(52) U.S. Cl. CPC A61C 17/10; A61C 17/092

(57) ABSTRACT

A dental mouthpiece can provide multiple features that can benefit both the patient and the doctor. The mouthpiece can take a generally flat configuration when not in use and may be resiliently folded into a C-shape for insertion into the patient's mouth. In some embodiments, the frame may be rigid or semi-rigid. The mouthpiece may provide retraction, suction ability, tongue control, airway and throat protection and a bite block in a single disposable or reusable device.

### BACKGROUND OF THE INVENTION

### 1. Field of the Invention

Embodiments of the invention relate generally to dental mouthpieces. More particularly, embodiments of the invention relate to a dental mouthpiece having features of retraction, suction, tongue guard, airway protection, suction options, and the like.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In performing dental procedures, there is often a need to perform multiple functions at the same time. Such functions can include keeping the patient's mouth open, retracting tissue such as a patient's lips, providing appropriate suction, and the like. Conventional lip and cheek retractors may be uncomfortable for users and do not provide any additional features or protections for the patient or doctor.

In view of the foregoing, there is a need for improved dental mouthpieces that can provide multiple features that can benefit both the patient and the doctor.

### SUMMARY OF THE INVENTION

Embodiments of the present invention provide a dental mouthpiece comprising lip retractor portions at opposing ends of the mouthpiece; a central region having a bite block member on each side thereof; cheek retractor portions connecting the lip retractor portions to the central region; at least one suction connector at the central region; and at least one suction opening disposed along a width of the central region, the at least one suction opening communicating with the at least one suction connector, wherein suction provided at the at least one suction connector provides a suction at the at least one suction opening.

Embodiments of the present invention further provide a dental mouthpiece comprising lip retractor portions formed as arcs at opposing ends of the mouthpiece; a central region having a bite block member on each side thereof; cheek retractor portions connecting each end of the arc of each of the lip retractor portions to the central region; at least one suction connector at the central region; at plurality of suction openings disposed in both a front side and a rear side along a width of the central region, the plurality of suction openings communicating with the at least one suction connector, wherein suction provided at the at least one suction connector provides a suction at the plurality of suction openings; and expansion and contraction elements disposed in the central region, the expansion and contraction elements permitting a width of the central region to be adjustable.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates front view of an anterior mouthpiece design according to an exemplary embodiment of the present invention;

FIG. 2 illustrates a back view of the mouthpiece design of FIG. 1;

FIG. 3 illustrates a side view of the mouthpiece design of FIG. 1;

FIG. 4 illustrates a side view, bent into an insertion/in use configuration, of the mouthpiece of FIG. 1;

FIG. 5 illustrates a front view of a semi-rigid frame anterior mouthpiece design according to an exemplary embodiment of the present invention;

FIG. 6 illustrates a rear perspective view of the mouthpiece of FIG. 5;

FIG. 7 illustrates a front view of a soft flexible material molded over a semi-rigid frame anterior mouthpiece design according to an exemplary embodiment of the present invention;

FIG. 8 illustrates a side view of the mouthpiece of FIG. 7;

FIG. 9 illustrates a side view, bent into an insertion/in use configuration, of the mouthpiece of FIG. 7;

FIG. 10 illustrates a front view of a mouthpiece according to an exemplary embodiment of the present invention;

FIG. 11 illustrates a back view of the mouthpiece of FIG. 10;

FIG. 12 illustrates a side view of the mouthpiece of FIG. 10;

FIG. 13 illustrates an end view of the mouthpiece of FIG. 10;

FIG. 14 illustrates a front view of the mouthpiece of FIG. 10 in a natural state; and

FIG. 15 illustrates a front view of the mouthpiece of FIG. 10 in a flexed state.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a dental mouthpiece that can provide multiple features that can benefit both the patient and the doctor. The mouthpiece can take a generally flat configuration when not in use and may be resiliently folded into a C-shape for insertion into the patient's mouth. In some embodiments, the frame may be rigid or semi-rigid. The mouthpiece may provide retraction, suction ability, tongue control, airway and throat protection and a bite block in a single disposable or reusable device.

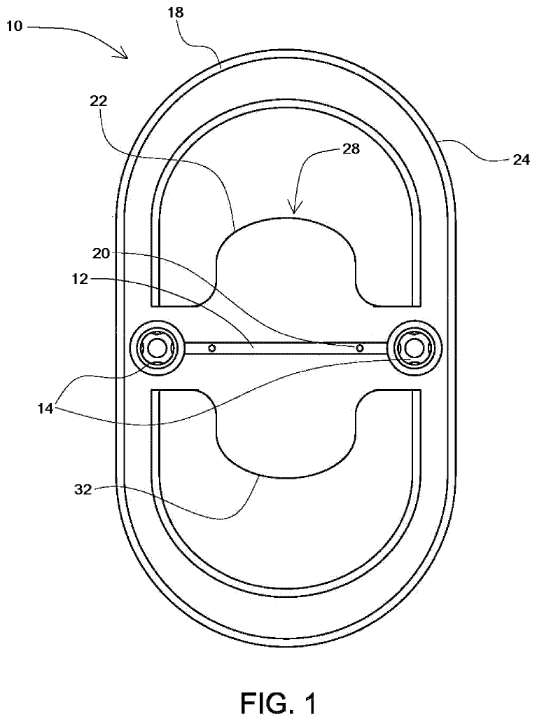

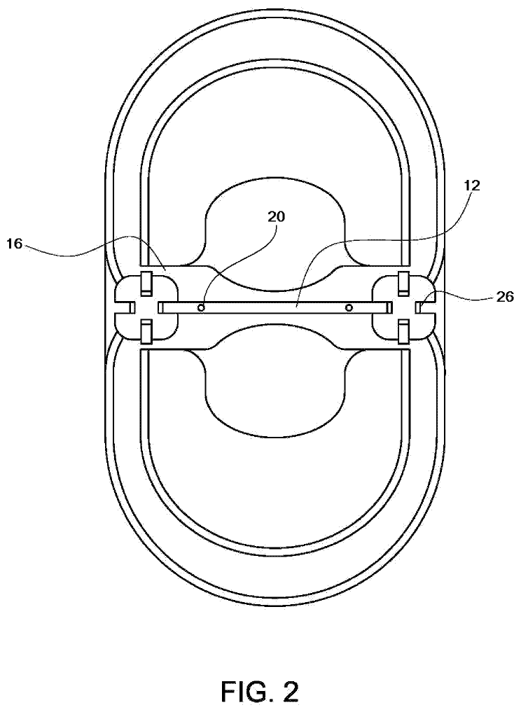

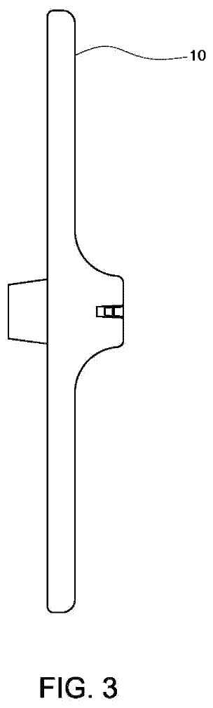

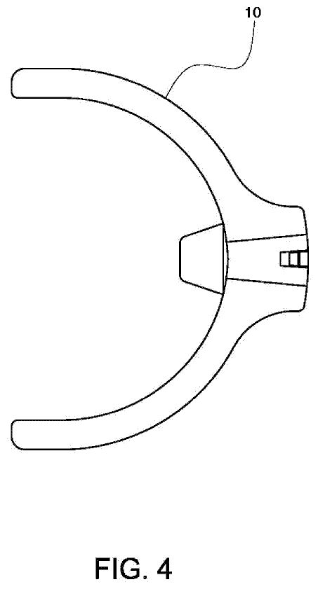

Referring now to FIGS. 1 through 4, according to a first embodiment of the present invention, a mouthpiece 10 can include suction channels 12, where, once the device is placed in the mouth, by bending the device into a C-shape, the mouthpiece 10 can create the suction channel 12 which provides suction when at least one of the suction ports 14 are connected to external suction. The suction can be provided, via the suction channels 12 at, for example, in the area of a bite block 16 and lip retractors 18.

Suction ports 20 communicate with the suction channel 12 so that saliva and excess irrigation fluid can be evacuated from the mouth and to provide inhibition from contaminating treatment areas of the oral cavity in the active treatment areas. Suction connector port 14 can include elastic materials to allow for connection with variety of sizes and designs of external suction sources. Suction can be provided in the airway protection 22, the bite block 16, the flexural support members 24, and the lip retractors 18, for example.

Suction openings 26 in the oral cavity have features to inhibit tissue from fully blocking suction. The suction openings 26 can provide suction to a buccal region of the patient when the mouthpiece is in place. When in use, the airway protection portion 22 and tongue shield 32 can keep a patient's tongue downward and out of the way. The user can rest their jaw on the bite block 16 while the lip retractors 18 can keep the lips out of the way from any procedures being performed by the dentist or hygienist, for example.

In the mouthpiece, the flexural resistance can assist in the insertion for various mouth morphologies where flexural support members 24 of the mouthpiece 10 open after folding for insertion and consistently self-seats the device in the maxillary and mandibular buccal vestibules of each patient.

The lip retractors 18 can extend outward from the central core of the device. These elements can include the flexural members which, when positioned, provide elevation and retraction for the labial surfaces both maxillary and mandibular. The counteractive forces of the maxillary and mandibular support members keep the lips retracted and away from treatment areas.

The molded flexural support members 24 and the lip retractors 18 provide upper lip, lower lip, and cheek retraction. This retraction provides unobstructed access and visibility for facial/labial areas of the mouth during dental procedures. The lips are prevented from touching gingival tissue and tooth structure. Lips and soft tissue are retracted and held away from dental tools for improved safety.

Flexural support members 24 can be joined to the central core 28 of the device by a molded connection 30. Gussets or fillets can be added to the flexural support member, thus positioning the bending center further away from the central core to create more retraction or lift to the lip retractor 4 as required.

A tongue shield 32 can inhibit patients from extending their tongue forward past the bite block 16, discouraging a patient from interfering during dental procedures and maintaining safety for the patient.

The airway protection portion 22 can extends up to the upper palatal vault and a throat protective shield 32 (also referred to as tongue shield 32) can extend to the floor of the mouth, thereby inhibiting debris from entering the patients throat and airway thus inhibiting accidental aspiration or ingestion of debris or equipment.

One or more external suction connection ports 14 allows for clinical access from multiple working positions without removal or reinsertion of the device into the oral cavity. While the device shown in FIGS. 1 through 4 include two such suction ports 14, the number and/or position of such ports may vary.

The bilateral bite block 16 can provide a place for patients to rest their jaw as well as providing an interincisal opening allowing for lingual, buccal, and occlusal access on the mandibular and maxillary arch.

The device of FIGS. 1 through 4 can provide an integrated retractor, bite block, tongue shield, airway shield and suction module in a disposable unit or in a non-disposable unit, depending on user needs and the specific application.

The entire device can be made of a soft material, similar to gingival tissue, to allow for comfort against the gums. For example, the device can be made from soft flexible materials, such as plastics, rubber, foams or the like. The device can include molded bumpers to allow the lip retractors to enhance patient comfort by inhibiting impingement directly against gingival tissue. In some embodiments, the bumpers may be formed from a sponge-like material to permit absorption of excess irrigation fluids from the oral cavity.

As described above, the device can be molded so that the device is substantially flat or semi-flat in a storage, non-use condition to allow for simple mold designs. The device can be resiliently confirmed into a C-shape bent configuration during oral insertion and during use thereof.

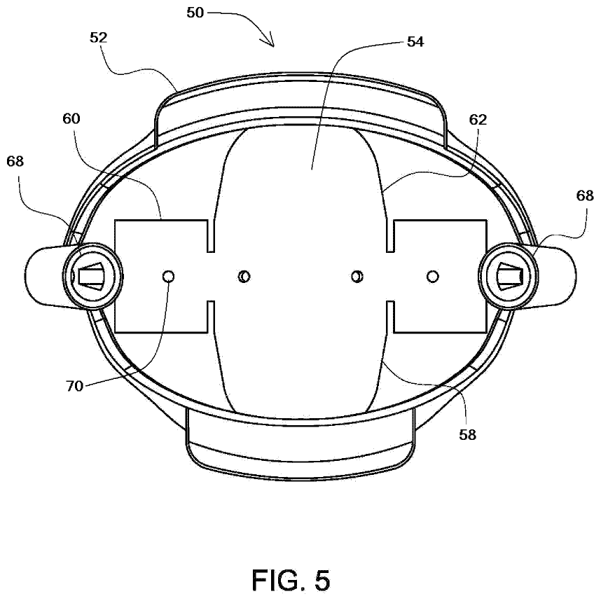

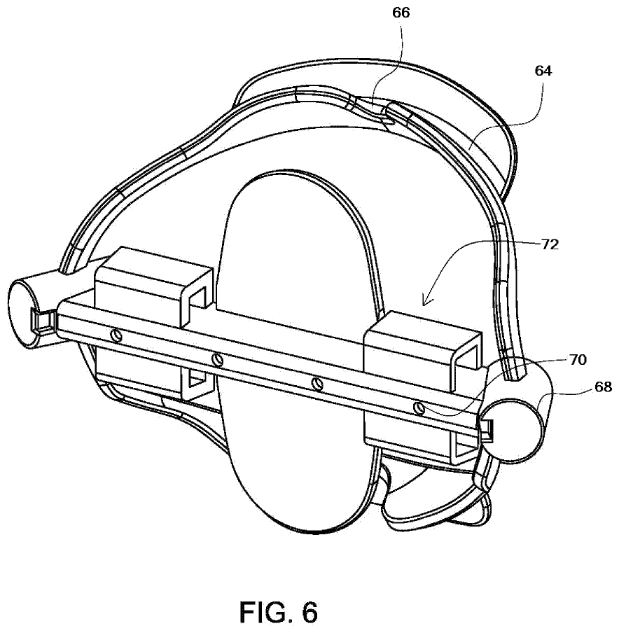

Referring now to FIGS. 5 and 6, a semi-rigid frame anterior mouthpiece 50 is shown. The features may be similar to many of those described above and as further discussed below.

Labial retraction can be provided by lip retractors 52 of the device that extend outward from the central core 54 of the device. These elements can include flexural members which, when positioned, provide elevation and retraction for the labial surfaces, both maxillary and mandibular. The counteractive forces of the maxillary and mandibular support members keep the lips retracted and away from treatment areas.

The molded flexural support members 56 provide upper lip, lower lip, and cheek retraction. This retraction provides unobstructed access and visibility for facial/labial areas of the mouth during dental procedures. The lips are prevented from touching gingival tissue and tooth structure. Lips and soft tissue are retracted and held away from dental tools for improved safety.

Flexural support members can be joined to the central core of the device by a molded connection. Gussets or fillets are added to the flexural support member positioning the bending center further away from the central core thus creating more retraction or lift to the lip retractor areas as required.

The tongue shield 58 can inhibit patients from extending their tongue forward past the bite block 60, discouraging a patient from interfering during dental procedures maintaining safety for patient.

The airway and throat protective shield 62 can extend up to the upper palatal vault and down to the floor of the mouth, thereby inhibiting debris from entering the patients throat and airway thus inhibiting accidental aspiration or ingestion of debris or equipment.

Molded retraction frame provides a self-seating lip saddle 64 that can guide the placement of the lips and inhibit them from becoming dislodged. Upper and lower cut out 66 can allow for frenum comfort.

The mouthpiece provides one or more suction ports 68 which, when connected to external suction provide a channel for saliva and excess irrigation fluids to be evacuated from the mouth and inhibits contaminating treatment areas of the oral cavity in the active treatment areas. One or more external suction connection ports 68 allow for clinical access from multiple working positions without removal or reinsertion of the device into the oral cavity. The suction openings 70 in the oral cavity can include features to inhibit tissue from fully blocking suction. Suction connector ports 68 can be formed from an elastic material to allow for connection with variety of sizes and designs of external suction sources.

Suction can be provided in the airway protection, bite block, flexural support members, lip retractors, suction channels, and external suction ports.

The bilateral bite blocks 60 provide a place for patients to rest their jaw as well as providing an interincisal opening allowing for lingual, buccal, and occlusal access on the mandibular and maxillary arch. The molded bite block module 72 is designed to be attached to the semi-rigid frame with a detailed mechanism feature which inhibits improper attachment and orientation. The molded bite block module 72/tongue shield unit 58 can connect to the frame orientation by various means, such as a feature that guides the connection and permits use of the mouthpiece with either the guard in the frame or removed from the frame and out of the patient's mouth.

The device provides a retractor, bite block, tongue shield, airway shield and suction module in a disposable unit or a non-disposable unit, depending on the particular application.

The device can include molded bumpers to allow the lip retractors to enhance patient comfort by inhibiting impingement directly against gingival tissue. In some embodiments, the bumpers may be formed from a sponge-like material to permit absorption of excess irrigation fluids from the oral cavity.

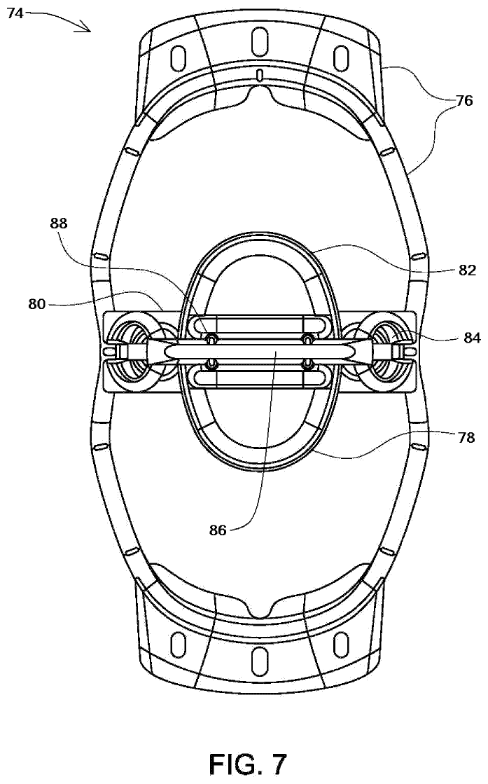

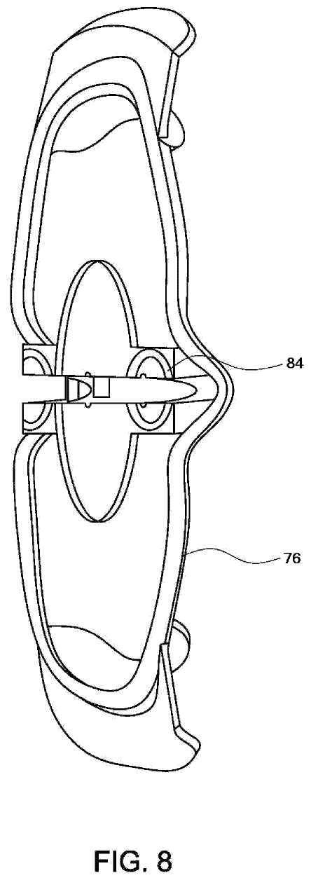

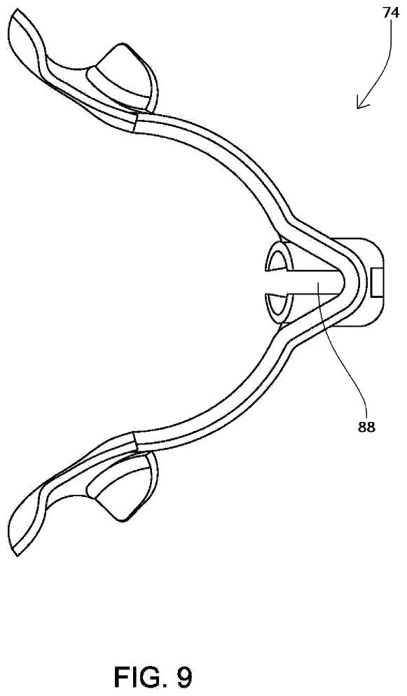

Referring now to FIGS. 7 through 9, a flexible material molded over a semi-rigid frame anterior mouthpiece system 74 is shown. The molded flexural support members 76 provide upper lip, lower lip, and cheek retraction. This retraction provides unobstructed access and visibility for facial/labial areas of the mouth during dental procedures. The lips can be prevented from touching gingival tissue and tooth structure. Lips and soft tissue can be retracted and held safely away from dental tools.

Labial retraction is provided by elements of the device that extend outward from the central core of the device, these elements include flexible members 76 which, when positioned in the use position, provide elevation and retraction for the labial surfaces both maxillary and mandibular. The counteractive forces of the maxillary and mandibular support members keep the lips retracted and away from treatment areas.

Support members will be joined to the central core of the device by a molded connection. Gussets or fillets are added to the support member positioning the bending center further away from the central core thus creating more retraction or lift to the lip retractor areas as required. Flexural resistance assists in the retraction for multiple mouth morphologies where flexural support members of mouthpiece open after folding for insertion and self-seats device in the maxillary and mandibular vestibules.

One or more materials are used to create a rigid back bone for retraction and a soft outer structure for comfort. Molded structure made of one or more dissimilar materials provide for retraction and patient comfort with minimal flexural members. For example, the mouthpiece can be made from a semi-rigid core over-molded with a soft, flexible material.

The device of FIGS. 7 through 9 may include features similar to that of the above structures, including those specifically described below. The tongue shield 78 can prevent patients from extending their tongue forward past the bite block 80, preventing a patient from interfering during dental procedures maintaining safety for patient.

The airway protection 82 can extend up to the upper palatal vault and down to the floor of the mouth which prevents debris from entering the patients throat and airways thus preventing accidental aspiration or ingestion of debris or equipment.

The bilateral bite blocks 80 provide a place to rest their jaw as well as providing an interincisal opening allowing for lingual, buccal, and occlusal access on the mandibular and maxillary arch.

The mouthpiece provides one or more suction ports 84 which, when connected to external suction, provides a channel 86 for saliva and excess irrigation fluids to be evacuated from the mouth. Once placed in the mouth, the mouthpiece will create suction channel 86 which provide suction when connected to external suction. Suction will be provided in the airway protection, bite block, flexural support members, suction channels, and suction ports.

The distributed suction openings 8 in the oral cavity have features to prevent tissue from fully blocking suction.

External suction connector port 84 can be formed from an elastic material to allow for connection with external suction device. One or more external suction connection ports 84 allow for clinical access from multiple working positions.

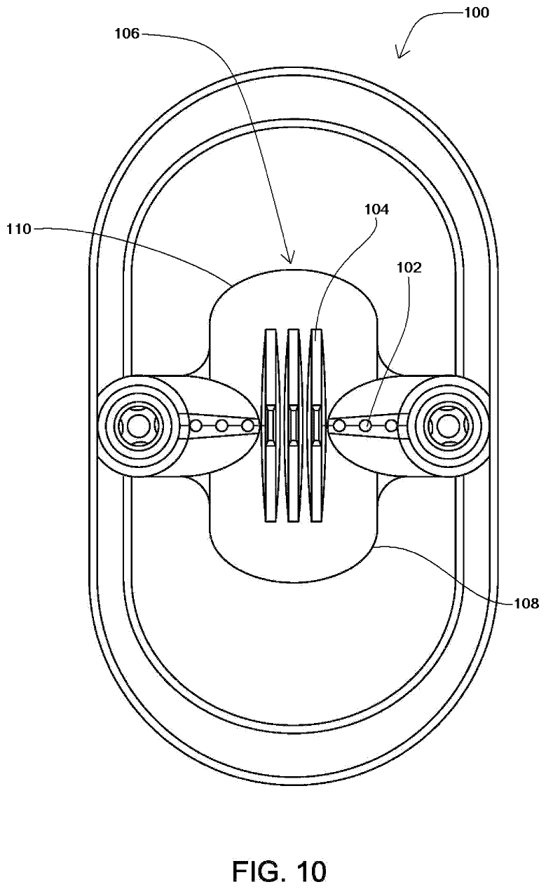

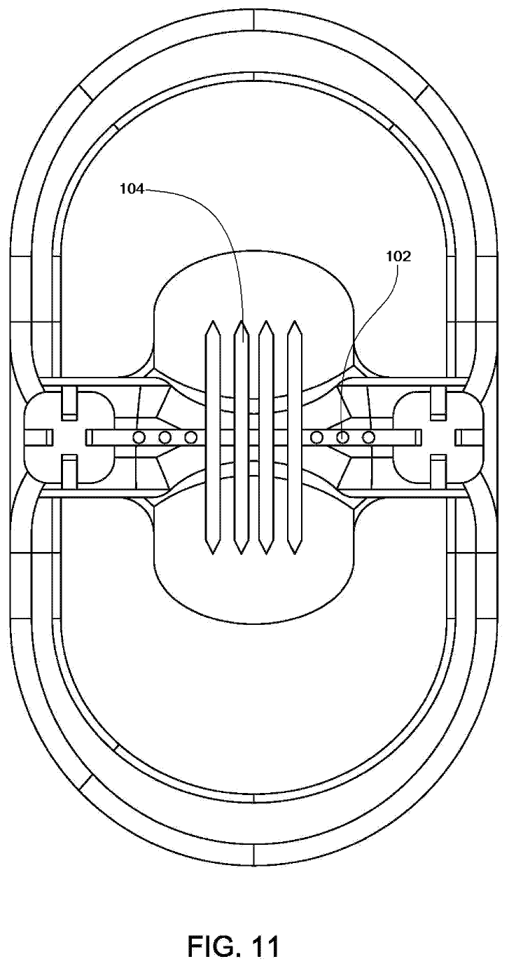

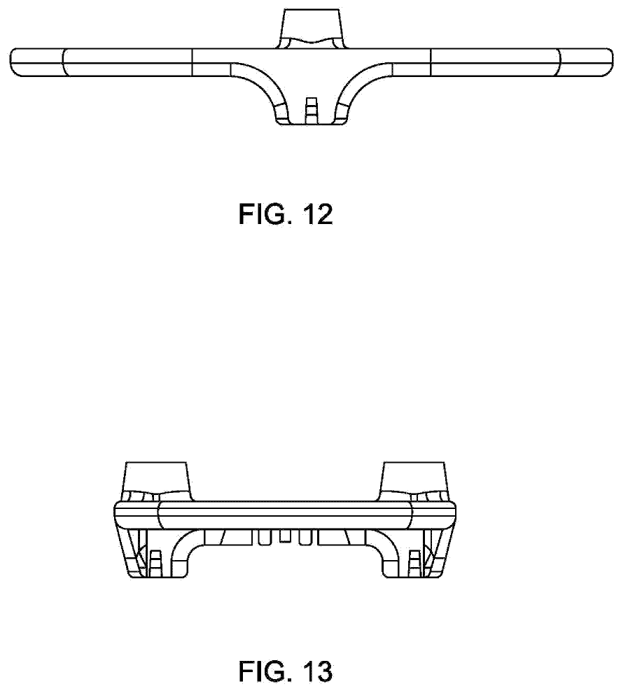

The embodiment of FIGS. 10-15 illustrate suction ports 102 along a width of the device 100. Such suction ports 102 can provide a negative pressure in the mouth of the patient, avoiding excess flow of droplets from the patient's mouth into the surrounding atmosphere. Such negative pressure may be especially useful for preventing the spread of virus from the patient to the dental professional.

The device 100 may provide features similar to the mouthpieces described above, where, when bent into a C-shape, it can be inserted into the patient's mouth to provide a bite block, lip and cheek retraction, and suction. The Embodiment of FIGS. 10-15 differ from that described above in that a central region 106, which includes a tongue guard 108 and an airway protection portion 110, has a plurality of thinned slots 104 formed therein.

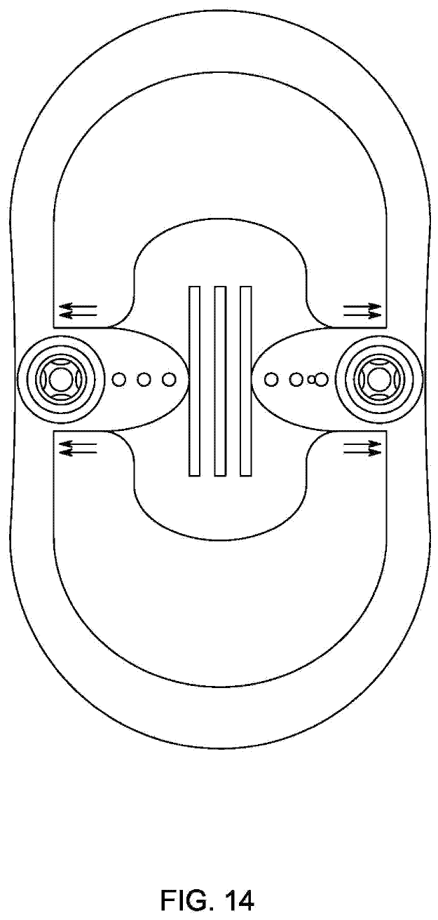

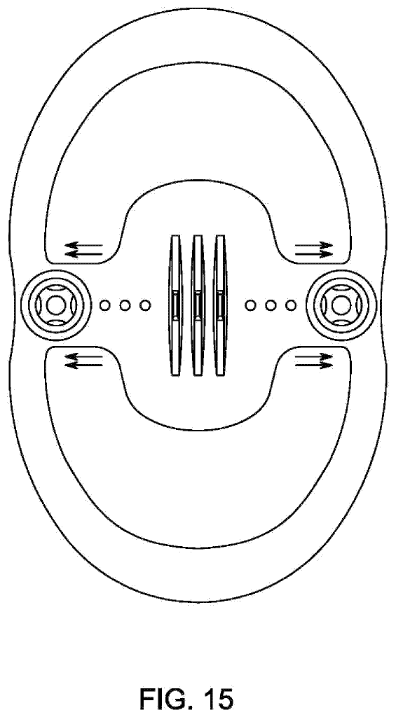

FIGS. 14 and 15 illustrate how the size of the device may be flexed to adjust to various sized oral cavities. The central region 106 may include regions of reduced thickness 104 to permit stretching outward or pressing inward of the sides of the device. In some embodiments, the reduced thickness regions may be slits through the material, but in other embodiments, as shown, the reduced thickness regions may be solid and reduced a given length and width to permit width-wise adjustability, as shown. Such adjustability may be from about 0.5 to about 3 cm, for example.

The devices described above provide retractor, bite block, tongue shield, airway shield and suction module in a disposable unit or a non-disposable unit, depending on the particular application.

The device can include molded bumpers to allow the lip retractors to enhance patient comfort by inhibiting impingement directly against gingival tissue. In some embodiments, the bumpers may be formed from a sponge-like material to permit absorption of excess irrigation fluids from the oral cavity.

The device can be molded so that the device is substantially flat or semi-flat in a storage, non-use condition to allow for simple mold designs. The device can be resiliently confirmed into a C-shape bent configuration during oral insertion and during use thereof. Such bending can close the channel, creating a tunnel for suction. Structure can flex to allow for external suction insertion and has flexural retraction to secure external suction in the mouthpiece.

The entire device can be made of a soft material similar to gingival tissue to allow for comfort against gums.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A dental mouthpiece comprising:
lip retractor portions at opposing ends of the mouthpiece;
a central region having a bite block member on each side thereof;
cheek retractor portions connecting the lip retractor portions to the central region;
at least one suction connector at the central region; and
at least one suction opening disposed along a width of the central region, the at least one suction opening communicating with the at least one suction connector, wherein suction provided at the at least one suction connector provides a suction at the at least one suction opening.

2. The dental mouthpiece of claim 1, further comprising a suction channel formed across the width of the central region when the dental mouthpiece is in use, the suction channel providing a flow path between the at least one suction connector and the at least one suction opening.

3. The dental mouthpiece of claim 1, wherein the lip retractor portions, cheek retractor portions and central region are coplanar in a non-use state of the dental mouthpiece.

4. The dental mouthpiece of claim 3, wherein the cheek retractor is flexible to permit the dental mouthpiece to fold into a C-shaped, use state thereof.

5. The dental mouthpiece of claim 1, wherein the central region includes a tongue guard extending downward toward a tongue of the patient during use of the dental mouthpiece.

6. The dental mouthpiece of claim 1, wherein the central region includes an airway protection portion extending upward during use of the dental mouthpiece.

7. The dental mouthpiece of claim 1, further comprising suction openings disposed at distal ends of the central region.

8. The dental mouthpiece of claim 1, further comprising expansion and contraction elements disposed in the central region, the expansion and contraction elements permitting a width of the central region to be adjustable.

9. The dental mouthpiece of claim 9, wherein the expansion and contraction elements are reduced thickness channels formed lengthwise in the central region.

10. The dental mouthpiece of claim 9, wherein the reduced thickness channels include at least one slot cut therethrough.

11. The dental mouthpiece of claim 1, further comprising lip saddles disposed on each of the lip retractors.

12. The dental mouthpiece of claim 11, further comprising a cutout in the lip saddle for frenum comfort during use of the dental mouthpiece.

13. A dental mouthpiece comprising:
lip retractor portions formed as arcs at opposing ends of the mouthpiece;
a central region having a bite block member on each side thereof;
cheek retractor portions connecting each end of the arc of each of the lip retractor portions to the central region;
at least one suction connector at the central region;
at plurality of suction openings disposed in both a front side and a rear side along a width of the central region, the plurality of suction openings communicating with the at least one suction connector, wherein suction provided at the at least one suction connector provides a suction at the plurality of suction openings; and
expansion and contraction elements disposed in the central region, the expansion and contraction elements permitting a width of the central region to be adjustable.

14. The dental mouthpiece of claim 13, wherein the expansion and contraction elements are reduced thickness channels formed lengthwise in the central region.

15. The dental mouthpiece of claim 13, wherein the reduced thickness channels include at least one slot cut therethrough.

16. The dental mouthpiece of claim 13, wherein the lip retractor portions, cheek retractor portions and central region are coplanar in a non-use state of the dental mouthpiece.

17. The dental mouthpiece of claim 16, wherein the cheek retractor is flexible to permit the dental mouthpiece to fold into a C-shaped, use state thereof.

18. The dental mouthpiece of claim 13, wherein the central region includes a tongue guard, extending downward toward a tongue of the patient during use of the dental mouthpiece, and an airway protection portion extending upward during use of the dental mouthpiece.
